(19) United States
(12) Patent Application Publication
(10) Pub. No.: US 2006/0024385 A1
(43) Pub. Date: Feb. 2, 2006

(54) Metabolic capacity enhancing compositions and methods for use in a mammal

(76) Inventor: Mark A. Pedersen, Kaysville, UT (US)

Correspondence Address: PATE PIERCE & BAIRD, 215 SOUTH STATE STREET, SUITE 550, PARKSIDE TOWER, SALT LAKE CITY, UT, 84111, US

(21) Appl. No.: 10/900,038

(22) Filed: Jul. 27, 2004

(51) Int. Cl. A61K 36/886; A61K 36/736

(52) U.S. Cl. 424/725; 424/766; 424/729; 424/770; 424/777; 424/732; 424/765; 424/744; 424/741

(57) ABSTRACT

Metabolic energy capacity enhancing compositions and methods for reducing oxidative stress and improving vitality in a mammal are disclosed. A composition for increasing metabolic energy capacity may be in a palatable liquid formulation or a solid dosage form and typically includes an anti-oxidant containing phytonectar and an energy catalyst. An anti-oxidant may include a polyphenol, anthrocyanin, bioflavonoid, proanthocyanidin, and a xanthone. An energy catalyst may include a mineral, vitamin, co-vitamin, carbohydrate and a lipid. In a presently preferred embodiment a composition includes phytonectar extracts from grape, aloe vera, apple, morinda citrifolia, scullcap, blueberry, prune, cranberry, elderberry, bilberry, and gentain and a mineral blend containing calcium, magnesium, manganese, zinc, chromium, selenium, iron, copper, molybdenum, vanadium, potassium, iodine, and cobalt. A method for increasing metabolic energy capacity in a mammal may include consuming a chemical component having the ability to undergo oxidation, producing free radicals and administering a composition having an anti-oxidant containing phytonectar and an energy catalyst.

### BACKGROUND

1. The Field of the Invention

The present invention relates generally to compositions and methods for improving vitality and more specifically it relates to compositions and methods for increasing energetic capacity of a mammal by neutralizing oxidative stress and providing energy catalysts.

2. The Background Art

As evidenced through historical records, mankind has used herbal and botanical tonics for a multitude of uses over several millennia. Early practitioners of the healing arts, especially those in Asia, the middle east, Africa, and eventually the people of the new world, relied upon observing the effects of various herbal preparations then applying this information to patients. From these observations, pharmacopoeia, compendia and other ledgers were kept to provide a knowledge base of natural products with nutritional, health, healing and medicinal properties.

Thermodynamically any reaction that releases energy rejects heat at a lower thermodynamic availability and discards waste products or byproducts. A transport process must exist to carry away these heat and mass byproducts. Otherwise, equilibrium is reached and the process stops.

As knowledge of the anatomy, physiology and metabolism of mammals has advanced, an interesting and somewhat ironic scientific principle has developed. It relates to the thermodynamics ofbiological processes. This principle may be called oxidative stress. Over the past several decades, observations and basic science studies have confirmed that while mammals and other animals are dependent upon oxygen for life, the by-products from oxygen consumption may result in several toxic compounds any of which may lead to significant tissue damage. These toxic compounds resulting as the by-products of oxygen consumption are sometimes referred to as “free radicals.” Moreover, it is widely believed that oxidation reactions within the body, and resulting free radical by-products may play an important role in aging and degenerative processes.

Aging and degenerative processes often manifest as conditions that may precipitate an inflammatory response in the body of a mammal. Inflammation involves the identification, removal and disposal of injured tissues. Mammals often associate injured tissues and the inflammation response with the perception of pain. There are numerous types of compounds involved in an inflammation process.

Mammals and other animals adapt to the inevitable onslaught of free radicals by producing enzymes (e.g., superoxide dismutase) and other compounds to destroy (e.g. recycle or discharge) free radicals or otherwise mitigate the damage they may cause. However, enzyme systems may be rate limited in their efficiency. In essence there may be a chronic or acute reduction in the capacity of the mammal to remove toxic compounds or waste products. As such, adaptive systems within mammals may be overwhelmed. In many instances mammals may have to draw upon other biochemical resources to confront the attack by free radicals. Most animals will crave and seek nutrients or other chemical compositions that are in short supply inbodily processes, as a result.

In addition to their direct effects, free radicals may also have significant indirect effects in mammals. Efforts to marshal biochemical resources from other areas of the body in order to meet the tissue damage being caused by free radicals may result in an overall decrease in metabolic efficiency in a mammal. In other words, strengthening defensive resources in one area (e.g., to combat neutralize, recycle, discharge, or otherwise free radicals) may ultimately weaken the overall integrity of the mammal. This may be especially important in the areas of energy production and general metabolism.

Metabolism involves the disposition of dietary nutrients and may be thought of as a collection ofbiochemical reactions and processes that occur in almost every cell in the body. Several types of metabolic reactions are known. Anabolic reactions (“anabolism”) may involve in the synthesis of compounds that contribute to body structure (e.g., muscles, bones, tissues and the like). Catabolic reactions (“catabolism”) may involve oxidative processes that release free-energy for use in other reactions and processes. Finally, amphibiotic reactions may be involved in multiple functions and multiple processes within the body of a mammal.

Reactions occurring in metabolic pathways and processes may often be mediated by enzymes (i.e., specialized proteins that catalyze a specific type of biochemical reaction), and accordingly may also require a co-enzyme (i.e., additional protein, often a metalloprotein, which may be required for a particular enzyme to function). Many enzymes and co-enzymes are known. Many enzymes and enzyme-related biochemical reactions may follow a complex pattern of kinetics (i.e., rate of change in a physical or chemical reaction). As such, enzyme systems may become “saturated” with reaction substrate. Due to other factors, enzyme systems may perform at less than optimal rates. These conditions may be referred to as rate limited conditions.

Many metabolic pathways may require a vitamin or mineral in order for metabolic reactions to proceed. Fluctuations in the availability of important vitamins or minerals may be another significant rate-limiting factor in metabolic reactions. Ultimately, reduced availability of vitamins or minerals may acutely impair metabolism and prevent optimal body functioning. In other words, there may be a reduction in the capacity of a mammal to produce or utilize energy. Moreover, this may become chronic, and may cause other related or permanent damage.

In some regions of the world, mild to moderate reduction in aging rates has occurred within certain societies and cultures. Those skilled in the art have attempted to evaluate and understand possible factors that may be responsible for slowing the aging process. In these investigations, it is worthwhile to note in the diet of the people who live longer and lead generally healthy lives whether they are ingesting some sort of novel herbal, botanical or mineral preparation.

Additional research into these herbal and botanical preparations has provided some understanding into a diverse group of chemical compounds, known broadly as anti-oxidants. As quantitative and analytical chemical techniques have progressed, there has been a somewhat parallel increase in the knowledge of anti-oxidant compounds. Anti-oxidant compounds are found in many plants, especially in fruits and vegetables of plants and may play a role in the texture, taste, smell and color of plants. Anti-oxidants within these herbals and botanicals may have a function similar to adaptive enzyme systems found in mammals, which is to destroy or deactivate free radicals. Those skilled in the art have sought to harvest the health utility of plant-contained anti-oxidants by the formulation and development of herbal tonic compositions.

An herbal tonic composition may include liquid extracts of fruits, vegetables, and other botanicals. Common components of herbal tonic compositions may include, for example, fruit extracts of Morinda citrifolia (“Noni”), and Mangosteen; sea vegetables, for example Kelp plant, Irish Moss plant, and Dulse plant; land vegetables including Garlic bulb, and Capsicum fruit; and botanicals including Ginseng root and Ginkgo biloba leaf.

Although there have been many advances in the formulation and delivery of herbal and botanical preparations, there are many problems that those skilled in the art have yet to overcome. One problem with conventional herbal tonic compositions is that the products do not contain standard potencies of anti-oxidant compounds, therefore resulting in variable effects after ingestion. Another problem with conventional herbal tonic compositions is that even though the use of singular fruits, vegetables, or botanicals has been reproduced and quantified, the anti-oxidant benefits of combining a variety of healing and energizing botanicals has been difficult to reproduce and quantify.

Still another problem with traditional herbal tonic compositions is that most botanicals have a bitter taste or unpalatable texture that require them to be placed in a solid dosage form such as a capsule or tablet in order to ensure adequate patient compliance. Yet another problem with traditional herbal tonic compositions is that the consumption of traditional solid dosage forms is decreasing as many consumers of natural products suffer from a phenomenon known as “pill fatigue.”

Another problem with traditional herbal tonic compositions is that they do not provide compounds specifically intended to combat oxygen free radicals in combination with compounds specifically intended to assist biochemical processes in the production and utilization of metabolic energy.

While some prior art formulations may be suitable for the particular purpose they address, they are not as suitable for providing a palatable botanical based liquid composition that acts as a tonic to relieve nutritional vacancies (e.g. absence, lack failure) that can lead to pain and inflammation and to reduce oxidative stress.

In addition, available prior art has not provided a formulation for reducing oxidative stress in a mammal while improving metabolic energy capacity and overall vitality in a mammal.

What is needed is a palatable botanical based composition with standardized anti-oxidant potencies and reproducible effects in reducing the effects of free radicals. What is also needed is a palatable botanical-based composition in the form of a liquid tonic to avoid reduced compliance due to pill fatigue. Moreover, what is further needed is such a composition with a waste product removal capacity enhancement component in combination with a metabolic energy capacity enhancement component for ingestion by a mammal.

In these respects, the herbal tonic compositions and methods for relieving the lack of energy processing nutrients as well as the waste-carrying nutrients limits the ability of the body to avoid consequent pain and inflammation. What is needed is a balance combination enhancing the production and utilization of energy while facilitating waste amelioration and repair of waste-induced damage to tissues. A substantial departure is needed from the conventional concept and design of the prior art. Balanced and substantially complete herbal tonic compositions and methods are needed for the enhanced removal of waste products, of energy production, to reduce their deleterious metabolic affects, and the enhanced levels of catalysts and other constituents for production and utilization of metabolic energy. Compositions and methods according to the invention reduce oxidative stress while improving overall vitality in a mammal.

### OBJECTS AND BRIEF SUMMARY OF THE INVENTION

In view of the foregoing, it is a primary object of the present invention to provide compositions and methods forreducing oxidative stress while improving overall vitality (e.g. improved and increased energy processing of given caloric foods) of a mammal.

It is another object of the present invention to provide compositions and methods for reducing oxidative stress and improving overall vitality of a mammal in the form of a liquid tonic.

It is a further object of the present invention to provide compositions and methods for reducing oxidative stress and improving overall vitality of a mammal containing standardized potencies of anti-oxidant and anti-inflammatory compounds.

In addition, it is an object of the present invention to provide compositions and methods for reducing oxidative stress and improving overall vitality of a mammal by providing a new and improved, palatable, liquid, ingestible to relieve inflammation in a variety of tissues in the body which may reduce pill fatigue associated with more conventional forms of administration.

It is a still further object of the present invention to provide compositions and methods for reducing oxidative stress and improving overall vitality of a mammal by providing a new and improved, palatable, liquid, ingestible composition to relieve pain in a variety of areas in the body.

Also, it is object of the present invention to provide compositions and methods for reducing oxidative stress and improving overall vitality of a mammal by providing a composition containing a compound which enhances cellular waste handling capacity while it enhances cellular capacity to produce and utilize metabolic energy.

Additionally, it is another object of the present invention to provide compositions and methods for reducing oxidative stress and improving overall vitality of a mammal in a delivery form which reduces the otherwise bitter taste of flavonoids and polyphenols contained in herbal and botanical compositions.

It is another object of the present invention to provide compositions and methods for reducing oxidative stress and improving overall vitality of a mammal that provides a method for developing and maintaining healthy blood sugar levels.

It is a further object of the present invention to provide compositions and methods for reducing oxidative stress and improving overall vitality of a mammal that provides a composition which includes only natural ingredients.

In addition, it is an object of the present invention to provide compositions and methods for reducing oxidative stress and improving overall vitality of a mammal that include a phytonectar comprising a reduction agent in an amount sufficient to reduce tissue damage caused by oxygen free radicals, and an energy catalyst in an amount effective to increase biochemical reactions in a mammal.

It is a still further object of the present invention to provide compositions and methods for reducing oxidative stress and improving overall vitality of a mammal that include a phytonectar with plant extracts selected from grape, aloe vera, apple, morinda citrifolia, scullcap, blueberry, prune, cranberry, elderberry, bilberry, gentain, orange, mango, kiwi, pomegranate, green tea, black tea, wheat, blackberry, raspberry, strawberry, onions, pear, cherries, plums, potato, tomato, grapefruit, pineapple, persimmon, eggplant, legume, coffee, guarana, cocoa bean, camellia leaf, kola nut, yerba mate, ginger root, evodia fruit, senna, pau d' arco bark, cascara sagrada, red clover, sheep sorrel, bayberry, boswellia gum, turmeric seed, mangosteen, perilla seed, ginseng root, root beer, stinging nettle leaf, podophyllum, bloodroot, myrrh gum, willow bark, pine bark, echinacea, goldenseal root and devil's claw root.

Also, it is an object of the present invention to provide compositions and methods for reducing oxidative stress and improving overall vitality of a mammal that include a phytonectar comprising a reducing agent selected from polyphenol, chelating agent, enzyme, carotenoid, sacrificial anti-oxidant and donor anti-oxidant.

Additionally, it is an object of the present invention to provide compositions and methods for reducing oxidative stress and improving overall vitality of a mammal that include a phytonectar comprising a polyphenol reduction agent (i.e., anti-oxidant) selected from phenolic acid, flavonoid, stilbene, lignan, xanthine, xingerone, xanthone, anthraquinone, caffeic acid, ferulic acid, tannin, gallic acid, flavone, flavonol, isoflavone, anthocyanin, anthocyanidin, flavanol, proanthocyanidin, flavanone, tannin, boswellic acid, ganistein, daidzein, catechin, baicalin, myricetin, quercetin, resveratrol, bromelain, luteolin, ginsenoside, salicin, pelargonidin, peonidin, cyanidin, delphinidin, malvidin, catechin, epicatechin, epigallocatechin, epicatechingallate and epigallocatechingallate.

It is another object of the present invention to provide compositions and methods for reducing oxidative stress and improving overall vitality of a mammal that include a phytonectar with plant extracts from grape, aloe vera, apple, morinda citrifolia, scullcap, blueberry, prune, cranberry, elderberry, bilberry and gentain.

It is a further object of the present invention to provide compositions and methods for reducing oxidative stress and improving overall vitality of a mammal that include (1) consuming an energy source material characterized by chemical component having the ability to undergo oxidation; (2) consuming a composition containing an anti-oxidant phytonectar and an energy catalyst blend; (3) oxidizing the energy source material and producing oxygen-based free radicals; and (4) enhancing free radical scavenging ability.

In addition, it is an object of the present invention to provide compositions and methods for reducing oxidative stress and improving overall vitality of a mammal that include (1) selecting an anti-oxidant phytonectar composition; (2) selecting an energy catalyst blend; (3) incorporating an anti-oxidant phytonectar and an energy-catalyst blend into a formulation suitable for delivering an effective amount of the anti-oxidants and energy catalysts to a mammal; (4) administering the formulation to a mammal; and (5) increasing energy delivery from the diet of the mammal while ameliorating oxidative stress consequent thereto.

It is a still further object of the present invention to provide compositions and methods for reducing oxidative stress and improving overall vitality of a mammal by providing a phytonectar capable of inhibiting the action of cyclooxygenase enzyme type 2 (COX-2).

Also, it is an object of the present invention to provide compositions and methods for reducing oxidative stress and improving overall vitality of a mammal by reducing the oxidative stress drivers for, and the inflammatory symptoms and effects associated with, arthritis (e.g., osteoarthritis, rheumatoid arthritis, leaky gout, gout, fibromyalgia, bursitis, atherosclerosis, chronic fatigue syndrome), cancer, cardiovascular disease (e.g., atherosclerosis, stroke, platelet aggregation, vascular and circulatory disease), diabetes, eye disease (e.g., retinopathy, cataracts, macular degeneration, loss of night vision), female health disorders (e.g., menopause, bone density disorders), male health disorders (e.g., prostate hypertrophy, prostate cancer), pain and respiratory disorders.

Additionally, it is an object of the present invention to provide compositions and methods for reducing oxidative stress and improving overall vitality of a mammal by reducing premature aging.

Consistent with the foregoing objects, and in accordance with the invention as embodied and broadly described herein, compositions and methods for reducing oxidative stress and improving overall vitality of a mammal are disclosed. One presently preferred embodiment of a composition in accordance with the invention may include a phytonectar comprising a reduction agent in an amount sufficient to significantly (e.g., measurably, objectively, sensibly to an individual) reduce tissue damage caused by oxygen free radicals, and an energy catalyst in an amount effective to increase biochemical reactions in a mammal. Specifically, one presently preferred embodiment in accordance with the invention may include a phytonectar with plant extracts selected from one or more of grape, aloe vera, apple, morinda citrifolia, scullcap, blueberry, prune, cranberry, elderberry, bilberry and gentain, and an energy catalyst mineral blend including calcium, magnesium, manganese, zinc, chromium, selenium, iron, copper, molybdenum, vanadium, potassium, iodine, cobalt, and other organic matter.

One presently preferred embodiment of a method for reducing oxidative stress and improving vitality in a mammal in accordance with the present invention may include (1) selecting a reduction agent containing phytonectar; (2) selecting an energy catalyst; (3) incorporating a phytonectar containing a reduction agent and a mineral composition containing an energy catalyst into a formulation suitable for delivering an effective amount of the composition to a mammal; and (4) administering the composition to a mammal. The method may optionally include observation of objective evidences of reduction of oxidative stress and increased energy delivery to the mammal, subjective observation of same, or both. The method may include feedback controlled administration of the phytonectar and mineral composition in accordance therewith.

### BRIEF DESCRIPTION OF THE DRAWINGS

The foregoing and other objects and features of the present invention will become more fully apparent from the following description, taken in conjunction with the accompanying drawings. Understanding that these drawings depict only typical embodiments in accordance with the invention and are, therefore, not to be considered limiting of its scope, the invention will be described with additional specificity and detail through use of the accompanying drawings in which:

FIG. 1 is a schematic block diagram illustrating one embodiment of a composition in accordance with the invention;

FIG. 2 is a schematic block diagram illustrating example embodiments of phytonectar compositions in accordance with the invention;

FIG. 3 is a schematic block diagram illustrating example embodiments of anti-oxidant compositions in accordance with the invention;

FIG. 4 is a schematic block diagram illustrating example embodiments of constituent catalysts in accordance with of the present invention;

FIG. 5 is a schematic block diagram illustrating examples of compositions of carrier components in accordance with the invention;

FIG. 6 is a schematic block diagram illustrating one embodiment of a process for increasing metabolic energy capacity in a mammal in accordance with the invention; and

FIG. 7 is a schematic block diagram illustrating one embodiment of a method for formulating a composition in accordance with the invention.

### DETAILED DESCRIPTION OF THE PREFERRED EMBODIMENTS

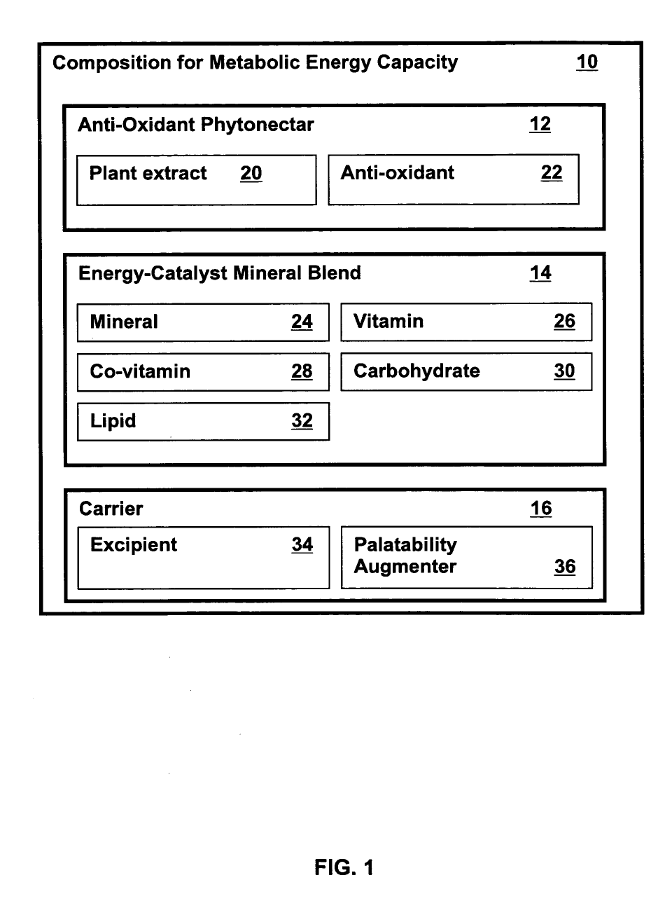

It will be readily understood that the components of the present invention, as generally described and illustrated in the Figures herein, could be arranged and designed in a wide variety of different configurations. Thus, the following more detailed description of the embodiments of systems and methods in accordance with the present invention, as represented in FIGS. 1 through 7, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

For the purposes of establishing definitional support for various terms used in the present application, Applicant provides the following technical comments and review.

The term “anthocyanin” may designate a soluble glycoside compound that often may serve as pigments in fruits, vegetables, leaves, flowers and other parts of the plant. Anthocyanins may be largely responsible for colors ranging from pink and purple to scarlet red and blue. Anthocyanins are soluble and upon hydrolysis they may yield anthocyanidins and sugars. Anthocyanins may be among the strongest biologically active compounds derived from plants and may have significant utility in nutrition to avoid eye and heart disorders, and also may have anti-inflammatory activity.

The term “anti-oxidant” may designate a compound that may inhibit oxidation of other compounds or tissues by oxygen or peroxides (“oxidants”). Anti-oxidants may often be used as preservatives for retarding the development of rancidity.

The term “bioflavonoid” may be used interchangeably with the term “flavonoid” and may designate a compound (e.g., such as rutin) having reducing or chelating properties in mammals (i.e., animals that are warm-blooded, contain skin that is generally covered with hair, give live birth to offspring, and typically nourish offspring with milk). A bioflavonoid may function as a pigment in some plants. Bioflavonoids are believed to have potent anti-inflammatory agents and may block the effect of histamine. Bioflavonoids may be the most abundant polyphenol in the diets of mammals. Bioflavonoids may have several subclasses including, but not limited to, flavones, flavonols, isoflavones, flavanols, and flavanones.

The term “capacity” may designate a measurement of volume or may generally designate an ability to store, process, treat, manufacture, or produce, and more particularly may designate a maximum processing, production, or output.

The term “extract” may designate separating or otherwise isolating a compound (e.g., chemical, molecule, mixture, etc.) from a substance (e.g., plant) by treating with a solvent (e.g., as alcohol), distilling, evaporating, applying pressure or centrifugal force, or by some other chemical or mechanical process.

The term “herbal” may designate a whole plant or any part of a plant characterized by having desirable nutritional, health, medicinal, savory, or aromatic properties.

The term “inflammation” may designate a response to tissue injury (e.g., as by infection or trauma) characterized by capillary dilatation, leukocytic infiltration, heat, and commonly pain. Inflammation may be a local response and in extreme circumstances may be acutely system (e.g., anaphylaxis) in a mammal. Inflammation may be responsible for the primary defense or control of noxious agents.

The term “metabolic energy” may be used interchangeably with “bioenergy” and may be expressed as a function of biochemical thermodynamics. Biochemical thermodynamics may describe heat transfer in a biochemical organism and may refer to the coupling of endergonic (i.e., requires work or expends energy) with exergonic (i.e., produces work or energy) reactions. Endergonic reactions may be involved in active transport across cell membranes, nervous system impulse transmission, synthesis of biomolecules (e.g., proteins, nucleic acids and the like), and muscular contraction. Exergonic reactions may be involved in the creation of high-energy compounds (e.g., adenosine triphosphate ATP and the like) for use in endergonic reactions.

The term “mineral” may designate a (e.g., generally solid or dissolved) homogeneous chemical element or compound (e.g., potassium, sodium, calcium, magnesium, phosphorous, iron, zinc, manganese, copper, cobalt, chromium, iodine, selenium, vanadium, molybdenum and the like) that results from the inorganic processes of nature.

The term “nectar” may be used synonymously with “phytonectar” and may designate a liquid beverage, typically made up of blended juices from fruit, vegetable or botanical sources. However, a nectar may include additional ingredients.

The term “oxidative stress” may refer to a condition where an excess of oxygen free radicals or a decrease in available anti-oxidant concentration or both may have caused tissue damage.

The term “pain” may designate mild, moderate or severe discomfort, which may be acute or chronic, generalized or localized and may typically result from being injured or hurt physically or mentally. In addition, pain may designate some dysfunction, derangement or lack of equilibrium in the physical or mental functions (e.g., resulting from a disease).

The term “polyphenol” may designate an organic compound having more than one phenol group. At least four types of polyphenols may be characterized, including, but not limited to, anthocyanins, bioflavonoids, proanthocyanins, and xanthones. Polyphenols may also include phenolic acids, stilbenes and lignans. Polyphenols may be found in many types of herbal and botanical sources and may work as a reducing agent (i.e., anti-oxidant) to protect an herbal or botanical from oxidative stress.

The term “polysaccharide” may designate a complex carbohydrate that may undergo hydrolysis to yield at least two molecules of monosaccharides (e.g., glucose). Polysaccharide may fall within the general chemical formula (C₆H₁₀O₅)x. Polysaccharides may include, for example and not by limitation, cellulose, starch and the like.

The term “proanthocyanidin” may designate a polymeric flavanol. They may also be referred to as oligomeric proanthocyanidins (OPCs). Typically OPCs may be found in bark, especially pine bark, and in grape seeds. Sometimes they may be referred to as “grape seed extracts.”

The term “xanthone” may designate a ketone C₆H₄(CO)(O)C₆H₄ that may form several natural yellow pigments (e.g., gentisin). Xanthones may be a bitter compound and may affect mood enhancement, blood sugar control, anti-inflammatory and anti-viral activity.

The term “vitality” may designate a capacity to live and develop and may be used synonymously with vigor. However, it means herein an increase in ability to expend or use energy; to be more energetic.

The physiology and metabolism ofmammals may be likened to the basic engineering principles of a combustion engine. In order for a combustion engine to generate power, oxygen and fuel (e.g., gasoline, diesel, etc. as fuel and air) are introduced into a chamber and an energy source (e.g., spark plug) precipitates a chemical reaction (i.e., combustion) of the fuel and oxygen to generate power. The power generated may commonly be used to propel a motor vehicle, but the power may be allotted to other functions as well.

The proper mixture of oxygen and fuel, together with appropriate timing on the application of a spark all contribute to maximizing the power (i.e., energy per unit of time) that an engine may produce. Many motor vehicles may require a periodic tune-up of their engine in order to optimize the engine capacity.

A mammal must take in fuel in the form of oxygen, water, carbohydrates, protein, fat and the like. This fuel may be turned into metabolic energy as carbohydrates are converted into carbon dioxide units. Much of this conversion may occur in the presence of specialized groups of enzymes (e.g., oxidoreductases). These enzymes may catalyze the production metabolic energy compounds. The capacity of a mammal to generate and produce metabolic energy may be highly dependent upon the quality and quantity of dietary caloric nutrients (i.e., metabolic fuels). Moreover, optimizing capacity to generate and utilize metabolic energy may be directly related to availability and mixture of all required dietary nutrients (i.e., lipids, protein, carbohydrates, vitamins, minerals, water and the like).

In many biochemical and metabolic reactions there may be a substrate, and at least one energy catalyst, typically an enzyme, and at least one enzymatic co-factor. The reaction rates (i.e., kinetics) of enzyme-catalyzed reactions may be characterized as saturable and rate-limited reactions. In general, if a substrate concentration is greater than that required for a corresponding enzyme concentration, the enzymes may become saturated. It may be said that the reaction is at maximum velocity. Enzyme kinetics are sometimes referred to as the “Michaelis-Menten” kinetic model.

Oxidative stress may initially appear to be a paradoxical condition in mammals and other animals. However, Applicant considers it a natural consequence of thermodynamics needing to be balanced. Oxygen is required as an essential fuel for producing and utilizing metabolic energy, yet the by-products of oxygen utilization results in the generation of waste products, free radical compounds, especially oxides and peroxides. Mammals and other animals have specialized enzymes (e.g., superoxide dismutase) to scavenge (i.e., destroy, recycle, carry away) free radicals.

In many mammals, the mixture of dietary nutrients may be such that an excess of free radicals may occur and the internal systems may be overwhelmed, rate-limited, or both. In other words, the capacity to handle free radicals may be maximized. This condition may make it possible for free radicals to accumulate in sufficient concentration to damage important tissues in the body of a mammal. Tissue damage may subsequently trigger an inflammation process resulting in the accumulation of inflammation cells and proteins designed to destroy and remove the damaged tissue. Tissue damage and an inflammation response may be perceived as pain. If the tissue damage is severe, a permanent and progressive condition may result.

Many tissues, organ systems, and conditions may be subject to oxidative stress and free radical damage. For example, and not by way of limitation, damage may occur and manifest in the immune system (e.g., allergies; cancer, including, but not limited to, bladder, breast, cervical, colorectal, lung melanoma, ovarian/endometrial, prostate, stomach, upper aerodigestive tract; rheumatoid arthritis; and other inflammatory conditions. It may also do so in the nervous system (e.g., Alzheimer's disease; Amyotrophic Lateral Sclerosis i.e., ALS, which may be classified as a motor neuron disease and sometimes referred to as “Lou Gehrig's Disease;” multiple sclerosis; muscular dystrophy; Huntington's Disease; Parkinson's Disease; schizophrenia; and tardive dyskinesia). It may likewise affect the cardiovascular system (e.g., heart disease; vascular disorders), ophthalmic system (e.g., cataracts, glaucoma, macular degeneration), metabolic system (e.g., Cystic Fibrosis, diabetes, diabetic neuropathy), and the digestive system (e.g., pancreatitis).

While a panacea for all of these conditions has yet to be found, ethnobotanists, pharmacognosists, and other natural product scientists have continued to look for herbal and botanical compounds that may be rich sources of anti-oxidant compounds. Analyzing the anti-oxidant power of herbal and botanical compositions may be accomplished with an oxygen radical absorbance capacity (ORAC) test or a hydroxy radical absorbance capacity (HORAC) test.

These tests may measure the free radical scavenging activity that exists in a particular compound or composition. An ORAC test value (measured in “Trolox” units) may provide a rough comparison to the anti-oxidant activity of vitamin E. Likewise, an HORAC test value (measured in “gallic acid” equivalents) may provide a rough comparison to the anti-oxidant activity of polyphenol compounds in protecting the body from free radical damage.

Herbal and botanical compositions may meet many anti-oxidant needs, but may not necessarily assist in maximizing metabolic energy capacity. In many enzyme-mediated biochemical processes a mineral or vitamin may be required in order for the reaction to proceed. Minerals and vitamins may be referred to as energy catalysts. Minerals that may be important to biochemical reactions include, but are not limited to, potassium, sodium, calcium, magnesium, phosphorous, iron, zinc, manganese, copper, cobalt, chromium, iodine, selenium, vanadium and molybdenum.

Vitamins important to biochemical reactions include, but are not limited to, vitamin A, vitamin C (e.g., ascorbic acid), vitamin D, vitamin E (e.g., tocopherols), vitamin B₁ (e.g., thiamine), vitamin B₂ (e.g., riboflavin), vitamin B₃ (e.g., niacin), vitamin B₅ (e.g., pantothenic acid), vitamin B₆ (e.g., pyridoxine), vitamin B₁₂ (e.g., cyanocobalamin) and folic acid (sometimes referred to as vitamin B₉). There may be many vitamin co-factors (i.e., co-vitamins or co-enzymes) that are important and often essential to metabolic energy reactions. Co-factors may include, for example and not by way of limitation, dinucleotides (e.g., flavin adenine dinucleotide (FAD), nicotinamide adenine dinucleotide (NAD), nicotinamide adenine dinucleotide phosphate (NADP)), thiamine pyrophosphate (TPP), coenzyme A (CoA), pyridoxal phosphate, methylcobalamin, and derivatives of tetrahydrofolate (THF), coenzyme Q10 (CoQ10) and biotin.

Reduced availability of a mineral, vitamin or co-vitamin may rate-limit a reaction and prevent maximum capacity in the production and utilization of metabolic energy. This may result in a mammal being unable to reach optimum vitality.

Referring now generally to FIGS. 1-5 and specifically to FIG. 1, a composition 10 for increasing metabolic energy capacity in a mammal may include an anti-oxidant phytonectar 12, a mineral blend containing an energy catalyst 14 and a carrier 16. A phytonectar 12 may include a plant extract 20 and an anti-oxidant 22. An energy catalyst 14 may be selected from a mineral 24, vitamin 26, co-vitamin 28, carbohydrate 30 and lipid 32. A carrier 34 may include at least one excipient 34 and a palatability augmenter 36. Metabolic energy may include, for example and not by way of limitation, reactions involving biochemical thermodynamics or heat transfer in a biochemical organism.

Metabolic energy may also refer to the coupling of endergonic (i.e., requires work or expends energy) with exergonic (i.e., produces work or energy) reactions. Endergonic reactions may be involved in active transport across cell membranes, nervous system impulse transmission, synthesis of biomolecules (e.g., proteins, nucleic acids and the like), and muscular contraction. Exergonic reactions may be involved in the creation of high-energy compounds (e.g., adenosine triphosphate ATP and the like) for use in endergonic reactions.

A mammal may be any animal that may be warm-blooded, contain skin that is generally covered with hair, give live birth to offspring, and typically nourish offspring with milk. A mammal may include, for example and not by way of limitation, a human, dog, cat, horse, cow, goat, sheep, ape, monkey, bear, elephant, tiger, lion and the like.

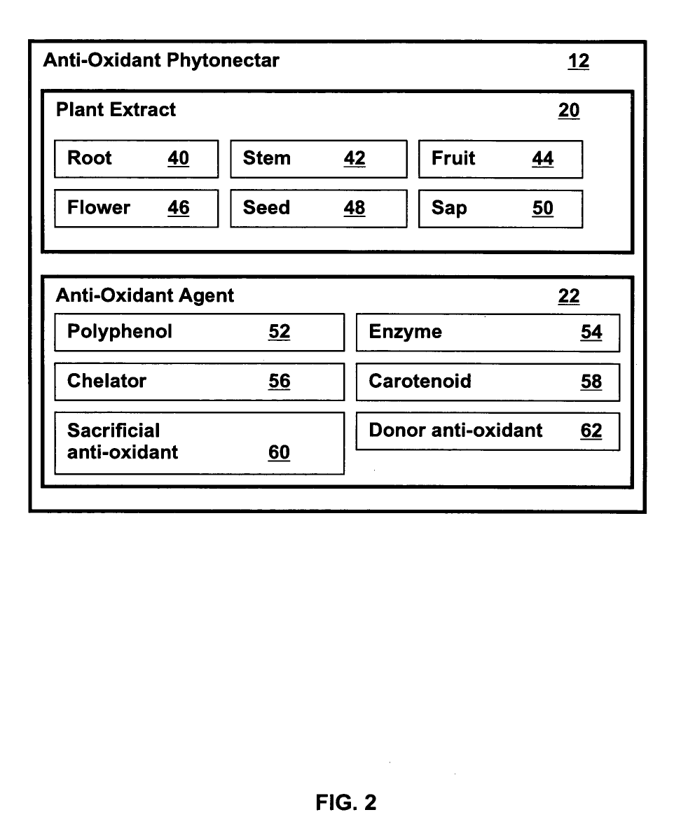

Still referring in general to FIGS. 1-5 and now specifically to FIG. 2, an anti-oxidant-containing phytonectar 12 may include a plant extract 20 and an anti-oxidant agent 22. A plant extract 20 may be selected from an entire plant or a plant portion such as root 40, stem 42, fruit 44, flower 46, seed 48 and sap 50. A plant extract 20 may be selected from selected from grape, aloe vera, apple, morinda citrifolia, scullcap, blueberry, prune, cranberry, elderberry, bilberry, gentain, orange, mango, kiwi, pomegranate, green tea, black tea, wheat, blackberry, raspberry, strawberry, onions, pear, cherries, plums, potato, tomato, grapefruit, pineapple, persimmon, eggplant, legume, coffee, guarana, cocoa bean, camellia leaf, kola nut, yerba mate, ginger root, evodia fruit, senna, pau d' arco bark, cascara sagrada, red clover, sheep sorrel, bayberry, boswellia gum, turmeric seed, mangosteen, perilla seed, ginseng root, root beer, stinging nettle leaf, podophyllum, bloodroot, myrrh gum, willow nark, pine bark, echinacea, goldenseal root and devil's claw root.

An anti-oxidant agent 22 may be capable of destroying or de-activating a free-radical compound. Classes of anti-oxidant agents 22 may include polyphenol 52, enzyme 54, chelating agent 56, carotenoid 58, sacrificial anti-oxidant 60 and donor anti-oxidant 62.

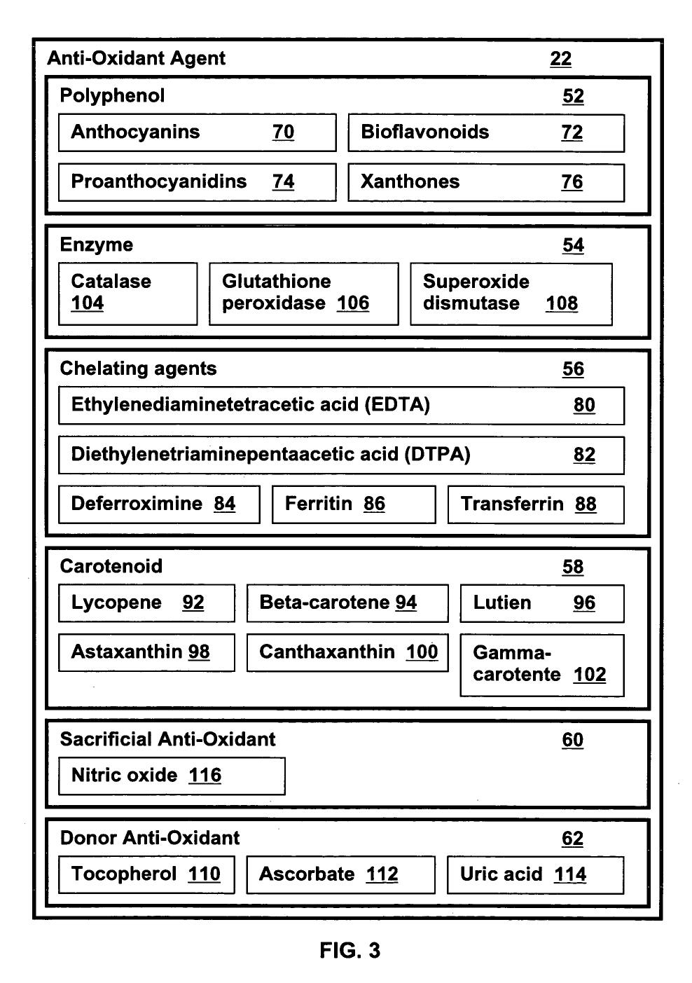

Now referring to FIG. 3 while continuing to refer generally to FIGS. 1-5, an anti-oxidant agent 22 may include a polyphenol 52 which may be organized into four general subgroups: anthocyanins 70, bioflavonoids 72, proanthocyanidins 74 and xanthones 76.

Anthocyanins 70 are a family of chemical compounds occurring in plants and may be responsible for color variation, especially pink, purple, red and blue. Anthocyanins 70 may be classified as glycoside compounds. Under certain conditions, a glycoside may be chemically separated into an aglycone and a sugar compound. Often, an aglycone may have similar properties to the parent glycoside compound. Anthocyanins 70 are known to be soluble in water and are believe to provide anti-oxidant protection to plants. Anthocyanins 70 may be found in high concentrations in grapes, bilberries, blueberries, elderberrires, cranberries and prunes. Anthocyanins 70 may be more stable in lower pH concentrations. In embodiments in accordance with the invention, pH is in a range of from about 3.8 to about 4.2.

Anthocyanins 70 are also known to have anti-inflammatory properties and therefore may be useful in supporting healthy brain function and the peripheral nervous system, skin and collagen function. In addition, anthocyanins 70 may provide nutritional support for diabetics by helping to reduce blood sugar levels. Anthocyanins 70 may also support healthy vascular function by protecting veins, arteries and capillaries from oxidative damage, plaque formation, or both. Anthocyanin 70 support of a healthy vascular system may also have benefits in the eye and extremities. Anthocyanins 70 may include, for example and not by way of limitation, pelargonidin, peonidin, cyanidin, delphinidin and malvidin.

Bioflavonoids 72 may be the most abundant anti-oxidant agent 22 occurring in the diets of many mammals, especially humans. Bioflavonoids 72 are know to occur in many plant extracts 20. In particular, bioflavonoids 72 are found in Chinese scullcap, gentain, cranberry and grape. Three important bioflavonoids 72 include biacalcin, myricetin and quercetin. Bioflavonoids 72 may have a role in color variation in plants. Bioflavonoids are also believed to have histamine blocking (i.e., anti-histamine or histamine antagonist) properties. Histamine blocking may be useful in alleviation allergy symptoms and inflammatory conditions, for example and not by way of limitation, arthritis.

Bioflavonoids 72 may work synergistically with other anti-oxidants, such as vitamin C, bromelain and nitric oxide. Bioflavonoids are believed to neutralize several types of free radicals, including, for example and not by way of limitation, nitric oxide, hydroxyl radical (i.e., HORAC), singlet oxygen, super-oxide radical (i.e., peroxynitrate radical). In addition to their anti-histamine properties, some bioflavonoids 72, especially biacalin, quercetin and myricetin, may block the function of cyclooxygenase type-2 enzyme (i.e., COX-2). COX-2 has recently become a major therapeutic target in the amelioration of several conditions, including, for example and not by way of limitation, arthritis, asthma, diabetes, stroke and other inflammation-mediated conditions.

Proanthocyanidins 74 may sometimes be referred to as condensed tannins or oligomeric proanthocyanidins (i.e., OPCs). Proanthocyanidins 74 are believed to be a polymer of flavanols. Some important proanthocyanidins 74 may include, for example and not by way of limitation, dimers, trimers and oligimers of catechin and epicatechin and their gallic acid esters (i.e., catechin (C), epicatechin (EC), epigallocatechin (EGC), epicatechin gallate (ECG), epigallocatechingallate (EGCG)).

Proanthocyanidins 74 are believed to have a significant anti-oxidant effect in ameliorating heart disease and cancer. Proanthocyanidins 74 have recently undergone extensive investigation in France in connection with studying the potential beneficial health effects of wine. Proanthocyanidins 74 may occur in high concentrations in grapes, bilberry, blueberry, cranberry, elderberry, prunes and apples. Proanthocyanidins 74 also may work independently of or synergistically with other anti-oxidants, especially vitamin E and tocopherols.

Xanthones 76 may have important anti-oxidant effects in the body, especially in connection with supporting nervous system function. Xanthones 76 may be found in high concentrations in gentain root. Xanthones 76 are believed to be among the most bitter compounds known. Xanthones 76 may have a role in ameliorating symptoms associated with mood disorders, depression and obesity. In addition, xanthones 76 may be useful in lowering blood sugar, reducing insulin resistance, combating viral infection and counteracting inflammation.

Chelating agents 56 may be used to deactivate metal ions, especially iron and copper, minimizing their potential to undergo oxidation reactions. Although many chelating agents 56 may only be available through interavenous administration, embodiments in accordance with the invention contemplate chelating agents 56 including, for example and not by way of limitation, ethylenediaminetetracetic acid (EDTA) 80, diethylenetriaminepentaacetic acid (DTPA) 82, deferroximine 84, ferritin 86, and transferrin 88.

A carotenoid 58 may also be used as an anti-oxidant agent 22. A carotenoid 58 may include lycopene 92, beta-carotene 94, lutien 96, astaxanthin 98, canthaxanthin 100 and gamma-carotene 102. An anti-oxidant 22 may include an enzyme 104 selected from catalase 104, glutathione peroxidase 106 and superoxide dismutase (SOD) 108. An anti-oxidant 22 may include a sacrificial anti-oxidant 60, such as nitric oxide or may include a donor anti-oxidant 62, for example and not by way of limitation, tocopherol 110, ascorbate (i.e., ascorbic acid) 112 and uric acid (i.e., urate) 114.

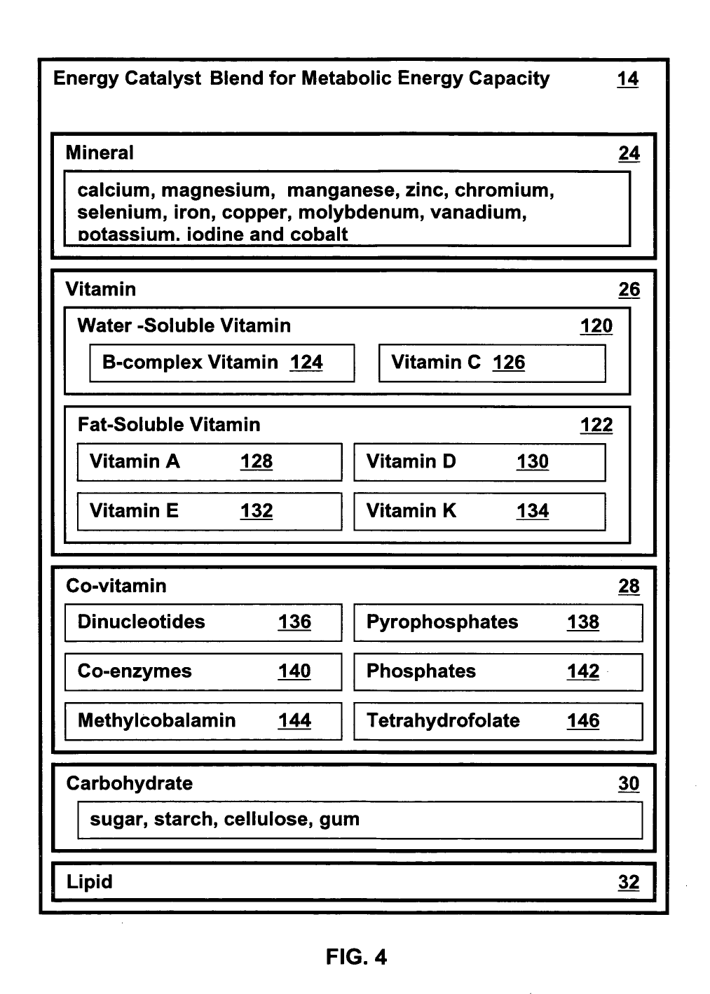

Referring now to FIG. 4 while continuing to refer generally to FIGS. 1-5, an energy catalyst for increasing metabolic energy capacity 14 may be selected from a mineral 24, vitamin 26, co-vitamin 28, carbohydrate 30 or lipid 32. An energy catalyst 14 may be essential or otherwise required in certain metabolic energy reactions in order to maintain a physiologically effective biochemical reaction rate. Compositions for increasing metabolic energy capacity in accordance with the present invention may increase the concentration of essential reaction components and thereby shift the bioenergetic equilibrium to a greater maximum reaction velocity by increasing it to an upper threshold for limiting the rate of enzymatic reactions.

A mineral 24 may be further selected from calcium, magnesium, manganese, chromium, zinc, selenium, iron, copper, molybdenum, vanadium, potassium, iodine and cobalt. A mineral 24 may have several roles in enhancing metabolic energy capacity and therefore increasing vitality in a mammal. For example, and not by limitation, a mineral 24 may be involved in active transport of compounds through cell membranes, may form the core of certain important metal-containing proteins (e.g., metalloproteins, metalloenzymes), may facilitate the action of a vitamin, and the like.

A vitamin 26 may be a water-soluble vitamin 120, for example and not by way of limitation a B-complex vitamin 124 (i.e., vitamin B₁ (e.g., thiamine), vitamin B₂ (e.g., riboflavin), vitamin B₃ (e.g., niacin), vitamin B₅ (e.g., pantothenic acid), vitamin B₆ (e.g., pyridoxine), vitamin B₁₂ (e.g., cyanocobalamin) and folic acid (sometimes referred to as vitamin Bg)) or vitamin C 126. A vitamin 26 may be a fat-soluble vitamin 122 selected from vitamin A 128, vitamin D 130, vitamin E 132 and vitamin K 134.

A co-vitamin 28 (i.e., co-factor or co-enzyme) may be important and often essential to metabolic energy reactions. A co-vitamin 28 may include, for example and not by way of limitation, a dinucleotide 136 (e.g., flavin adenine dinucleotide (FAD), nicotinamide adenine dinucleotide (NAD), nicotinamide adenine dinucleotide phosphate (NADP)), pyrophosphate 138 (e.g., thiamine pyrophosphate), co-enzyme 140 (e.g., co-enzyme A (CoA), co-enzyme Q10 (CoQ10)), phosphate 142 (e.g., pyridoxal phosphate), methylcobalamin 144, tetrahydrofolate 146. A co-vitamin 28 may work synergistically with a vitamin 26 or interact with a vitamin 26 in a manner to activate it.

A carbohydrate 30 may be important in metabolic energy capacity by storing energy in mammals. Carbohydrates 30 may be defined a compound from a group of organic compounds that includes sugars, starches, celluloses, and gums. Carbohydrates 30 may often be found in the dietary intake ofmammals and other animals. Carbohydrates 30 may contain only carbon (C), hydrogen (H), and oxygen (O), usually in the ratio 1:2:1, respectively.

A lipid 32 may include fats, waxes, phosphatides and related compounds. A lipid 32 may be the basic building block of cell structures and may be essential the vitality of a mammal.

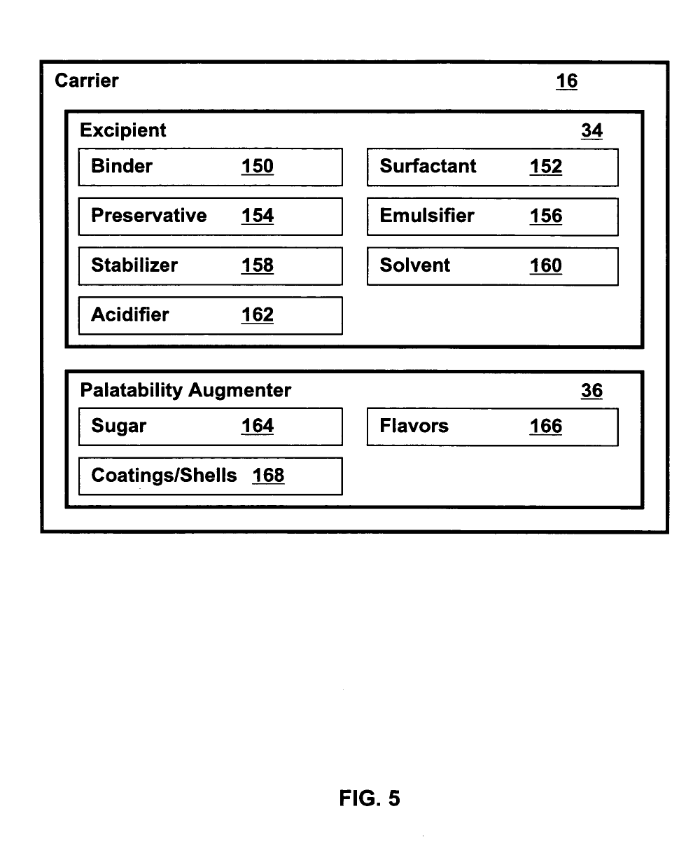

Still generally referring to FIGS. 1-5 and referring now specifically to FIG. 5, a carrier 16 may include at least one excipient 34 and at least one palatability augmenter 36. An excipient 34 may be selected from binder 150, surfactant 152, preservative 154, emulsifier 156, stabilizer 158, solvent 160 and acidifier 162. Representative compounds from these excipients 34 may be selected to mollify a particular pharmaceutical properties of an example embodiment in accordance with the invention. Pharmaceutical properties may include, for example and not by way of limitation, pH, suspendability, shelf-life, rancidity and the like.

A palatability augmenter 36 may be selected from sugar 164, flavors 166 and coatings 168 in order to mollify the taste, smell, texture or appearance of an example embodiment in accordance with the invention.

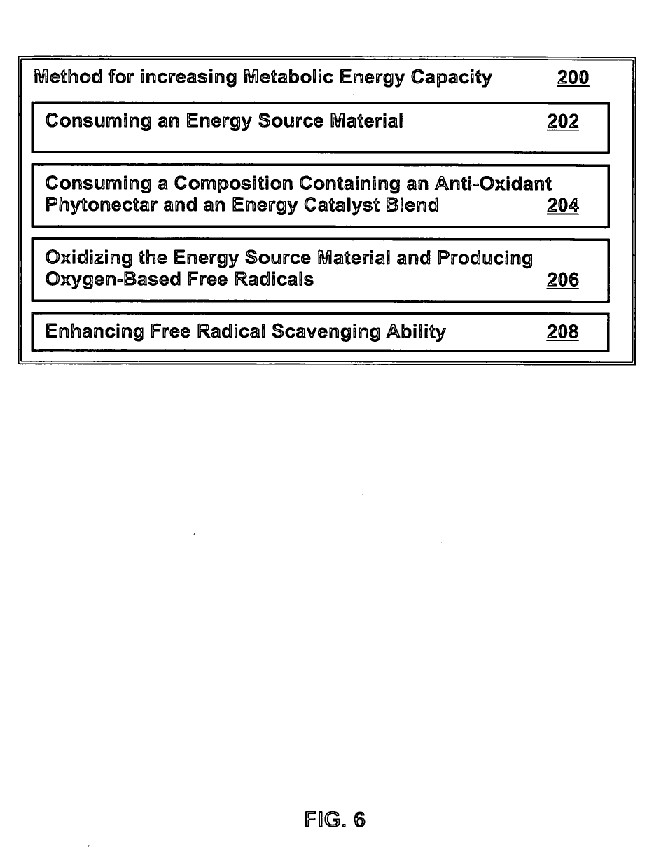

Referring now to FIG. 6, a method 200 for increasing metabolic energy capacity in a mammal may be broadly defined as: (1) consuming 202 an energy source material characterized by chemical component having ability to undergo oxidation; (2) consuming 204 a composition containing an anti-oxidant phytonectar and an energy catalyst blend; (3) oxidizing 206 the energy source material and producing oxygen-based free radicals; and (4) enhancing 208 free radical scavenging ability in a mammal. An energy source material may be any material that serves as a fuel for biochemical reactions, for example, and not by way of limitation, oxygen, protein, carbohydrates, fats, lipids, water or any combination thereof.

A method 200 for increasing metabolic energy capacity in a mammal may provide for increasing waste handling capacity of a mammal by providing an increased concentration of anti-oxidant compounds capable of providing additional scavenging of free radicals. In addition, a method 200 for increasing metabolic energy capacity in a mammal may further provide energy catalysts that may be important or otherwise essential to facilitating increased reaction rates of bioenergetic processes.

Because an equilibrium shift may occur with a method 200 for increasing metabolic energy capacity, a mammal may require less effort to marshal important chemical compounds to defend or otherwise prevent tissue damage by free radicals. Moreover, the more efficient utilization of internal resources may therefore lead to enhancing the vitality of a mammal.

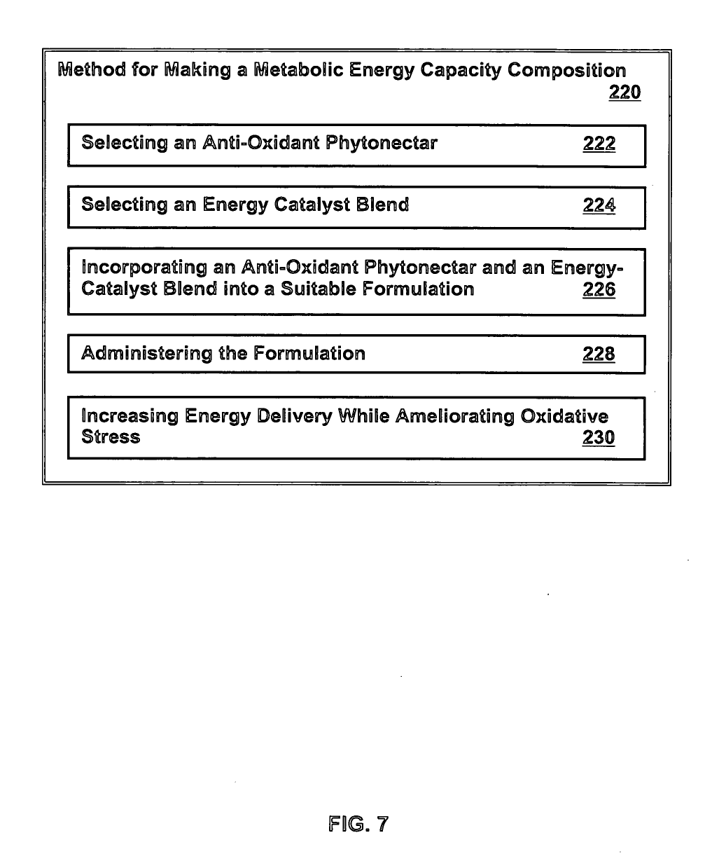

Referring now to FIG. 7, a method 220 for making a composition for increasing metabolic energy capacity in a mammal may include: (1) selecting 222 an anti-oxidant phytonectar composition; (2) selecting 224 an energy catalyst blend; (3) incorporating 226 an anti-oxidant-containing phytonectar and an energy-catalyst blend into a formulation suitable for delivering an effective amount of the anti-oxidant agents 22 and energy catalysts to a mammal; (4) administering 228 the formulation to a mammal; and (5) increasing 230 energy delivery from the diet of the mammal while ameliorating oxidative stress consequent thereto.

The following examples will illustrate the practice of the present invention in further detail. It will be readily understood by those skilled in the art that the following metabolic energy capacity increasing compositions and methods in accordance with the embodiments generally described and illustrated in the Examples herein, are to be viewed as exemplary of the principles of the present invention, and not as restrictive to a particular structure or process for implementing those principles. Thus, the following more detailed description of the methods, formulations and compositions in accordance with the present invention, as represented in the following Examples, is not intended to limit the scope of the invention, as claimed, but is merely representative of selected exemplary embodiments in accordance therewith.

### EXAMPLE I

### Arthritis Formula

Based on the foregoing description a metabolic energy capacity enhancing composition and method may include a phytonectar with plant extracts from grape, aloe vera, apple, morinda citrifolia, scullcap, blueberry, prune, cranberry, elderberry, bilberry and gentain and an energy catalyst blend including the following minerals: calcium, magnesium, manganese, zinc, chromium, selenium, iron, copper, molybdenum, vanadium, potassium, iodine and cobalt. A composition for enhancing metabolic energy capacity may be summarized as follows:

phytonectar:

| Ingredient | Range % (w/w) |
|---|---|
| Whole Grape Nectar | 10-30 |
| Grape Seed extract | 0.5-4 |
| Bilberry extract | 0.5-4 |
| Morinda citrifolia extract | 0.5-4 |
| Blueberry extract | 0.5-4 |
| Elderberry extract | 0.5-4 |
| Cranberry extract | 0.5-4 |
| Chinese Scullcap root extract | 0.1-0.5 |
| Gentian root extract | 0.05-0.2 |
| Aloe vera juice | 10-30 |
| Whole apple nectar | 10-30 |
| Purified Water | 30-50 |
| Mineral essence | 0.2-0.5 |
| Flavors | qs |

Energy Catalyst Blend:

| Ingredient | Range % (w/w) |
|---|---|
| Calcium | 1.4 |
| Magnesium | 1.12 |
| Manganese | 0.0049 |
| Zinc | 0.0091 |
| Chromium | 0.00042 |
| Selenium | 0.0014 |
| Iron | 0.021 |
| Copper | 0.00084 |
| Molybdenum | 0.00028 |
| Vanadium | 0.00042 |
| Potassium | 42 |
| Iodine | 0.00042 |
| Cobalt | 0.007 |
| Organic Matter¹ | 55.43422 |

¹Organic matter consists of polyphenols, chlorides, amino acids, and weak organic acids (e.g., citrates, etc.).

The total dissolved solids may be about 500 parts per million (ppm). A sufficient quantity of mineral blend may be added to one (1) liter of water, which is about 500 mg/liter. The concentrations are expressed in w/w which may be defined as percent by weight and may define the percent by weight of the component in the solution. Typically, w/w may designate grams of the component in 100 grams of solution.

In one embodiment in accordance with the present invention, grape may be a source of antioxidant polyphenols that reduce inflammation and oxidative stress as described above. Grapes may be rich in protective nutrients from the tannin family of anti-oxidants. The polyphenols in grapes may be powerful enough that they may withstand the fermentation process. Anti-oxidants in grape may also relieve inflammation and may have use in relieving joint pain and nervous tension. Grape extracts may make up between about five percent (5%) to about twenty percent (20%) of a presently preferred embodiment of a composition for increasing metabolic energy capacity. Grape extracts may be produced by carefully selecting ripe, purple grapes, washing and crushing the grapes and grinding the seeds and pulp to produce a nectar. The nectar is standardized to a minimum proanthocyanin content of about four percent (4%).

Scullcap root may be a source of flavonoids that may reduce inflammation, pain and oxidative stress as described above. In traditional Chinese medicine, baical skullcap (Scutellaria baicalensis) may be prescribed for many conditions. Scullcap may support healthy liver function and reducing inflammation associated with premenstrual syndrome. Scullcap may also be used to relieve the effects of nervous tension, insomnia, and stress related nervousness. Recent research on the flavonoids found in Scullcap (biscalein) may suggest that they are powerful cyclo-oxygenase-type 2 (COX-2) inhibitors. Scullcap may therefore have benefit in inflammatory conditions including joint and bowel health. Scullcap extracts may make up between about five percent (5%) to about twenty percent (20%) of a presently preferred embodiment of a composition for increasing metabolic energy capacity. Scullcap extracts may be produced by carefully selecting clean, dry roots of Scutellaria baicalensis. The roots may be crusted and ground into a pulp with the addition of purified mineral water. The scullcap extract thus produced may be standardized to a minimum baicalein content of about thirty percent (30%).

Gentain root extract may serve as a source of xanthones that may enhance elimination and improve digestion as described above. It has been traditionally used as an aperitif, laxative, and blood purifier. Gentain extract may be a common botanical in many alcoholic bitters. Gentain active principals may be among the most bitter compounds known. A presently preferred embodiment of a composition in accordance with the present invention

In one presently preferred embodiment of the present invention, an important feature of a metabolic energy capacity enhancing composition may be its ability to incorporate these bitter substances into a palatable form. The xanthone bitter principals in Gentain root may have many functions similar to the bitter principals in Aloe and Cascara sagrada bark. Gentain nectar makes up between about two percent (2%) to about twenty percent (20%) of a composition in accordance with preferred embodiments of the present invention.

Gentain extract may be produced by carefully selecting clean dry roots of Geniana purpurea. The roots may be crushed and ground into a pulp with the addition of purified mineral water. The nectar thus produced is then standardized to a minimum xanthone content of about five percent (5%).

Bilberry may server as a source of anthocyanins that reduce oxidative stress as described above. Bilberry, sometimes referred to as huckleberry, or whortleberry may be a European variety of Blueberry that may have been used in traditional medicine since the middle ages. Studies may show that Bilberry supports the vascular system. Bilberry extracts may contain flavonoids and highly colored anthocyanins that support capillary strength and flexibility, thin the blood and support the release of vasodilators. Anthocyanins may be powerful antioxidants that support healthy blood pressure control. Bilberry extracts also may contain glocoquinine which may support healthy blood sugar levels. Bilberry extracts may be especially useful in issues related to eye health and more specifically impaired night vision. Bilberry extract may make up between about five percent (5%) to about twenty percent (20%) of a composition in accordance with presently preferred embodiments of the present invention. Bilberry extracts may be produced by carefully selecting ripe Bilberries, washing and crushing the berries and grinding them to a pulp to produce a nectar. Bilberry extract may be standardized to contain a minimum anthocyanin content of about five percent (5%).

Blueberry extract may server as a source of anthocyanins that may reduce oxidative stress as described above. Blueberries may be a close cousin of Bilberries and have been traditionally used for the same purposes as Bilberries. Additionally, Blueberries may also support urinary tract health and to improve appetite and energy. Blueberries may contain antioxidant anthocyanins and protective flavonoids that may make up as much as ten percent (10%) of the Blueberry. Blueberry extract may make up between about five percent (5%) to about twenty(20%) of a composition in accordance with presently preferred embodiments of the present invention. Blueberry extract may be produced by carefully selecting ripe Bilberries, washing and crushing the berries and grinding them to a pulp to produce a nectar. The nectar may be standardized to contain a minimum anthocyanin content of about five percent (5%).

Prune extract may serve as a source of anthocyanins that may reduce oxidative stress as described above. Prune extract may also be a source of dietary fiber to promote bowel regularity. Prunes obtained from dried plums may contain anthocyanins that may provide one of the highest antioxidant potentials of any fruit. In addition to its antioxidant potential, Prune extract may also be a rich source of dietary fiber that promotes regularity. Prunes may be desirous for their gentle, bowel cleansing action. Prune extract may make up between about five percent (5%) to about twenty percent (20%) of a composition in one presently preferred embodiment in accordance with the present invention. Prune extract may be produced by carefully selecting ripe, dried Plums, washing and crushing them and grinding to a pulp with the addition of mineral to produce a nectar. The nectar may be standardized to contain a minimum anthocyanin content of about five percent (5%).

Elderberry extract may serve as a source of anthocyanins that may reduce oxidative stress as described above. Elderberries have traditionally been used to improve skin complexion, and for a variety of respiratory ailments. Elderberry extract may contain both anthocyanins and flavonoids that may contribute to their ability to reduce inflammation and pain. Anthocyanins may be potent antioxidant compounds. Elderberry extract may make up between about five percent (5%) to about twenty (20%) of a composition in one presently preferred embodiment in accordance with the present invention. Elderberry extract may be produced by carefully selecting ripe Elderberries, washing and crushing the berries and grinding them to a pulp to produce a nectar. The nectar may be standardized to contain a minimum anthocyanin content of about four percent (4%).

Morinda citrifolia extract (“Noni” extract) may serve as a source of bromelain precursors that reduce inflammation and improve digestion. Morinda citrifolia extract may contain polysaccharides and polyphenols, including flavonoids, that may provide support for healthy joint and soft tissue function. A primary action of Morinda citrifolia may be to provide a precursor to bromelain. Bromelain may be a digestive enzyme and anti-inflammatory agent. Morinda citrifolia extract may make up between about five percent (5%) to about twenty percent (20%) of a composition in one presently preferred embodiment in accordance with the present invention. Morinda extract may be produced by carefully selecting ripe fruit, washing and crushing the fruit and grinding them to a pulp to produce a nectar. The nectar may be standardized to contain a minimum of three percent (3%) Bromelain activity.

Cranberry extract may serve as a source of anthocyanins and flavonoids that reduce inflammation and oxidative stress as described above. Cranberries have been a folk-remedy for centuries. Cranberries may have traditionally been used to support urinary tract health. The anthocyanins and flavonoids in Cranberries may have antiseptic, anti-inflammatory, and antioxidant properties. Studies may also show that Cranberries may be beneficial to all mucous membranes of the body. Cranberry extract may make up between about five percent (5%) to about twenty percent (20%) of a composition in one presently preferred embodiment in accordance with the present invention. Cranberry extract may produced by carefully selecting ripe fruit, washing and crushing the fruit and grinding them to a pulp to produce a nectar. The nectar may be standardized to contain a minimum of four percent (4%) quinic acid.

Apple extract may serve as a source of anthocyanins and may reduce oxidative stress as described above. Apples may have been a source of constitutional strength for centuries.

Apples may contain antioxidant anthocyanins and dietary fiber that may promote regularity. Apple extract may make up between about five percent (5%) to about twenty percent (20%) of a composition in one presently preferred embodiment in accordance with the present invention. Apple extract may be produced by carefully selecting ripe fruit, washing and crushing the fruit and grinding them to a pulp to produce a nectar. The nectar may be standardized to contain a minimum of about 0.5% anthocyanins.

Aloe Vera extract may serve as a source of polysaccharides that may reduce oxidative stress and inflammation. Aloe Vera may have many plant healing utilities. It is variously known as “lily of the desert”, and the “plant of immortality.” Extensive research into its healing properties may have shown it to contain polysaccharides (e.g., acemannan) and bitter principals that produce most of its healing effects. The polysaccharides may form slick gels that sooth the skin and digestive tract. The bitter anthroquinines may promote peristalsis and may enhance bowel elimination. An additional use for aloe may be as an immune stimulant. Aloe Vera extract may make up between about five percent (5%) to about twenty percent (20%) of a composition in one presently preferred embodiment in accordance with the present invention. Aloe vera extract may be produced by carefully selecting ripe leaves, washing and crushing the leaves and grinding them to a pulp to produce a nectar. The nectar may be standardized to contain a minimum of about 0.5% polysaccharide.

A composition for reducing oxidative stress and improving vitality in a mammal in accordance with a presently preferred embodiment of the present invention may be analyzed for anti-oxidant capacity with ORAC and HORAC laboratory methods and compared to other anti-oxidant compositions. The results may be summarized in the following table.

TABLE 1

| | | | Anthocyanin |
|---|---|---|---|
| Composition | ORAC¹ | HORAC² | content³ |
| Example I | 1,245 | 104 | 17.00 |
| Tahitian Noni | 416 | 19 | 0.12 |
| Xango | 499 | 34 | 0.50 |
| Sea Silver | 96 | 6 | not detected |

¹micromoles Trolox equivalent per fluid ounce
²Gallic acid equivalent per fluid ounce
³milligrams per fluid ounce

After preparing the extracts and weighing the correct proportions, a composition in one presently preferred embodiment of the present invention may be prepared by blending the eleven (11) anti-oxidant containing phytonectar extracts together with an energy catalyst mineral blend and adding sufficient preservatives, such as sodium benzonate and potassium sorbate, to insure the microbial stability of the product. Shelf stability may be further enhanced by adding a sufficient food grade acid to the composition to bring the pH of the composition into a range of between about 3.5 and 4.2. The resulting composition may be placed in a glass or polymer bottle and capped to produce a shelf stable product. Alternatively, a liquid composition in one presently preferred embodiment in accordance with the present invention may be converted to a solid dosage form such as a tablet or capsule by drying the nectars and blending them into equal portions.

Additionally, a liquid composition in one presently preferred embodiment in accordance with the present invention may also be incorporated into a powder or other food-like delivery system.

A composition in one presently preferred embodiment in accordance with the present invention may be designed to be orally consumed in a dose of from about one (1) to about six (6) tablespoons taken with one or more of the main meals of the day. A composition in one presently preferred embodiment in accordance with the present invention may be taken orally. It may be mixed with other food or water. The dose may be continued indefinitely to maintain vitality or the dose may be increased to meet the demands of various health concerns. A composition in one presently preferred embodiment in accordance with the present invention may be safe for people of all ages. For children under the age of twelve (12) years the dosage may be reduced to about one (1) tablespoon or less using similar time intervals between doses.

### EXAMPLE II

Joint Inflammation and Pain

| Ingredient | Amount/daily dose | |
|---|---|---|
| Glucosamine HCl powder | 1500 | mg |
| Myrrh gum powder | 100 | mg |
| Aloe vera juice | 10000 | mg |
| Boswellia serrata herb powder | 100 | mg |
| Cranberry juice | 10000 | mg |
| Natural flavor | 500 | mg |
| Sodium benzoate | 60 | mg |
| Potassium sorbate | 60 | mg |
| Citric acid | 120 | mg |
| Fructose | 3000 | mg |
| Glycerine | 600 | mg |
| Xanthan gum | 75 | mg |
| Purified water | 33885 | mg |

This combination of nutrients is particularly useful for treating ailments associated with joint inflammation and pain. The recommendation for use is to take two (2) ounces by mouth per day. Example II may optionally include the mineral blend as defined in Example I.

### EXAMPLE III

Oral Mucosa Inflammation

| Ingredient | Amount/daily dose | |
|---|---|---|
| Rutin sulfate, sodium salt | 50 | mg |
| Lemon juice | 1000 | mg |
| Green tea leaf powdered extract | 400 | mg |
| Chinese Scullcap root powder | 50 | mg |
| Aloe vera juice | 10000 | mg |
| Arabinogalactan from Larch bark | 100 | mg |
| Apple juice | 10000 | mg |
| Natural flavor | 500 | mg |
| Sodium benzoate | 60 | mg |
| Potassium sorbate | 60 | mg |
| Citric acid | 20 | mg |
| Fructose | 3000 | mg |
| Glycerine | 600 | mg |
| Xanthan gum | 75 | mg |
| Purified water | 34150 | mg |

This combination of nutrients is particularly useful for treating ailments associated with inflammation of the gums and mouth including apthous ulcers, cankers, gingivitis, periodontitis, cold sores, etc. The recommendation for use is once daily to swish two (2) ounces in the mouth for one (1) minute and swallow. Example III may optionally include the mineral blend as defined in Example I.

### EXAMPLE IV

Blood Glucose Control

| Ingredient | Amount/daily dose | |
|---|---|---|
| Blueberry juice | 1000 | mg |
| Bilberry juice | 1000 | mg |
| Cranberry juice | 10000 | mg |
| Prune juice | 1000 | mg |
| Hawthorne berry powder | 150 | mg |
| Pomegranate fruit powder | 100 | mg |
| Grape juice | 10000 | mg |
| Aloe vera juice | 1000 | mg |
| Apple juice | 10000 | mg |
| Natural flavor | 500 | mg |
| Sodium benzoate | 60 | mg |
| Potassium sorbate | 60 | mg |
| Citric acid | 120 | mg |
| Glycerine | 600 | mg |
| Xanthan gum | 60 | mg |
| Purified water | 24350 | mg |

This combination of nutrients is particularly useful for maintaining healthy blood sugar levels in cases of diabetes and hypoglycemia. The recommendation for use is to take two (2) ounces by mouth once daily. Example IV may optionally include the mineral blend as defined in Example I.

### EXAMPLE V

Cholesterol Control

| Ingredient | Amount/daily dose | |
|---|---|---|
| Blueberry juice | 1000 | mg |
| Bilberry juice | 1000 | mg |
| Prune juice | 1000 | mg |
| Hawthorne berry powder | 150 | mg |
| Pomegranate fruit powder | 100 | mg |
| Blood Orange fruit powder | 100 | mg |
| Grape juice | 10000 | mg |
| Aloe vera juice | 10000 | mg |
| Green tea leaf powder | 400 | mg |
| Natural flavor | 500 | mg |
| Sodium benzoate | 60 | mg |
| Potassium sorbate | 60 | mg |
| Citric acid | 120 | mg |
| Fructose | 3000 | mg |
| Glycerine | 600 | mg |
| Xanthan gum | 60 | mg |
| Purified water | 31850 | mg |

This combination of nutrients is particularly useful for maintaining healthy cholesterol levels and for reducing inflammation in the cardiovascular system. The recommendation for use is to take two (2) ounces by mouth once daily. Example V may optionally include the mineral blend as defined in Example I.

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. A composition for increasing metabolic energy capacity in a mammal, the composition comprising:
a phytonectar comprising an anti-oxidant agent in an amount effective to reduce tissue damage caused by oxygen free radicals;
an energy catalyst in an amount effective to increase biochemical reactions in a mammal; and
a carrier suitable for delivering an effective amount of an anti-oxidant-containing phytonectar and energy catalyst to a mammal.

2. The composition as defined in claim 1, further comprising a form selected from the group consisting of liquid, tablet, capsule, powder, granule, spray, ointment and cream.

3. The composition as defined in claim 1, wherein the phytonectar further comprises a plant extract selected from the group consisting of root, stem, fruit, flower, seed and sap.

4. The composition as defined in claim 1, wherein the phytonectar further comprises an extract from a plant selected from the group consisting of grape, aloe vera, apple, morinda citrifolia, scullcap, blueberry, prune, cranberry, elderberry, bilberry, gentain, orange, mango, kiwi, pomegranate, green tea, black tea, wheat, blackberry, raspberry, strawberry, onions, pear, cherries, plums, potato, tomato, grapefruit, pineapple, persimmon, eggplant, legume, coffee, guarana, cocoa bean, camellia leaf, kola nut, yerba mate, ginger root, evodia fruit, senna, pau d' arco bark, cascara sagrada, red clover, sheep sorrel, bayberry, boswellia gum, turmeric seed, mangosteen, perilla seed, ginseng root, root beer, stinging nettle leaf, podophyllum, bloodroot, myrrh gum, willow bark, pine bark, echinacea, goldenseal root and devil's claw root.

5. The composition as defined in claim 1, wherein the phytonectar comprises plant extracts from grape, aloe vera, apple, morinda citrifolia fruit, Chinese scullcap root, blueberry, prune, cranberry, elderberry, bilberry and gentain root.

6. The composition as defined in claim 1, wherein the anti-oxidant agent is selected from the group consisting of polyphenol, chelating agent, carotenoid, enzyme, donor anti-oxidant and sacrificial anti-oxidant.

7. The composition as defined in claim 6, wherein the anti-oxidant agent further comprises a polyphenol member selected from the group consisting of phenolic acid, flavonoid, stilbene, lignan, xanthine, xingerone, xanthone, anthraquinone, caffeic acid, ferulic acid, tannin, gallic acid, flavone, flavonol, isoflavone, anthocyanin, anthocyanidin, flavanol, proanthocyanidin, flavanone, tannin, boswellic acid, ganistein, daidzein, catechin, baicalin, myricetin, quercetin, resveratrol, bromelain, luteolin, ginsenoside, salicin, pelargonidin, peonidin, cyanidin, delphinidin, malvidin, catechin, epicatechin, epigallocatechin, epicatechingallate and epigallocatechingallate.

8. The composition as defined in claim 1, wherein the energy catalyst is selected from the group consisting of mineral, vitamin, co-vitamin, carbohydrate and lipid.

9. The composition as defined in claim 8, wherein the mineral is selected from the group consisting of calcium, magnesium, manganese, zinc, chromium, selenium, iron, copper, molybdenum, vanadium, potassium, iodine and cobalt.

10. The composition as defined in claim 8, wherein the energy catalyst comprises calcium, magnesium, manganese, zinc, chromium, selenium, iron, copper, molybdenum, vanadium, potassium, iodine and cobalt.

11. The composition as defined in claim 8, wherein the vitamin is selected from the group consisting of B-complex Vitamins, Vitamin C, Vitamin A, Vitamin D, Vitamin E, Vitamin K and co-enzymes.

12. The composition of claim 11, wherein B-complex Vitamins are selected from the group consisting of Vitamin B₁ (thiamine), Vitamin B₂ (riboflavin), Vitamin B₃ (niacin), Vitamin B₅ (pantothenic acid), Vitamin B₆ (pyridoxine), Vitamin B₉ (folic acid), and Vitamin B₁₂ (cyanocobalamin).

13. The composition as defined in claim 1, wherein the carrier comprises at least one excipient and at least one palatability augmenter.

14. A composition for increasing metabolic energy capacity in a mammal, the composition comprising:
a phytonectar comprising plant extracts from grape, aloe vera, apple, morinda citrifolia fruit, Chinese scullcap root, blueberry, prune, cranberry, elderberry, bilberry and gentain root comprising an anti-oxidant agent in an amount effective to reduce tissue damage caused by oxygen free radicals;
an energy catalyst mineral blend comprising calcium, magnesium, manganese, zinc, chromium, selenium, iron, copper, molybdenum, vanadium, potassium, iodine and cobalt in an amount effective to increase biochemical reactions in a mammal; and
a carrier suitable for delivering an effective amount of an anti-oxidant-containing phytonectar and energy catalyst to a mammal.

15. A method for increasing metabolic energy capacity in a mammal, the method comprising:
consuming an energy source material characterized by chemical component having the ability to undergo oxidation;
consuming a composition containing an anti-oxidant phytonectar and an energy catalyst blend;
oxidizing the energy source material and producing oxygen-based free radicals; and
enhancing free radical scavenging ability.

16. The method as defined in claim 15, wherein administering a composition further comprises a form selected from the group consisting of liquid, tablet, capsule, powder, granule, spray, ointment and cream.

17. The method as defined in claim 15, wherein the phytonectar further comprises an extract from a plant selected from the group consisting of grape, aloe vera, apple, morinda citrifolia, scullcap, blueberry, prune, cranberry, elderberry, bilberry, gentain, orange, mango, kiwi, pomegranate, green tea, black tea, wheat, blackberry, raspberry, strawberry, onions, pear, cherries, plums, potato, tomato, grapefruit, pineapple, persimmon, eggplant, legume, coffee, guarana, cocoa bean, camellia leaf, kola nut, yerba mate, ginger root, evodia fruit, senna, pau d' arco bark, cascara sagrada, red clover, sheep sorrel, bayberry, boswellia gum, turmeric seed, mangosteen, perilla seed, ginseng root, root beer, stinging nettle leaf, podophyllum, bloodroot, myrrh gum, willow bark, pine bark, echinacea, goldenseal root and devil's claw root.

18. The method as defined in claim 15, wherein the phytonectar comprises plant extracts from grape, aloe vera, apple, morinda citrifolia fruit, Chinese scullcap root, blueberry, prune, cranberry, elderberry, bilberry and gentain root.

19. The method as defined in claim 15, wherein the anti-oxidant agent is selected from the group consisting ofpolyphenol, chelating agent, carotenoid, enzyme, donor anti-oxidant and sacrificial anti-oxidant.

20. The method as defined in claim 15, wherein the anti-oxidant agent further comprises a polyphenol member selected from the group consisting ofphenolic acid, flavonoid, stilbene, lignan, xanthine, xingerone, xanthone, anthraquinone, caffeic acid, ferulic acid, tannin, gallic acid, flavone, flavonol, isoflavone, anthocyanin, anthocyanidin, flavanol, proanthocyanidin, flavanone, tannin, boswellic acid, ganistein, daidzein, catechin, baicalin, myricetin, quercetin, resveratrol, bromelain, luteolin, ginsenoside, salicin, pelargonidin, peonidin, cyanidin, delphinidin, malvidin, catechin, epicatechin, epigallocatechin, epicatechingallate and epigallocatechingallate.

21. The method as defined in claim 15, wherein the energy catalyst is selected from the group consisting of mineral, vitamin, co-vitamin, carbohydrate and lipid.

22. The method as defined in claim 21, wherein the mineral is selected from the group consisting of calcium, magnesium, manganese, zinc, chromium, selenium, iron, copper, molybdenum, vanadium, potassium, iodine and cobalt.

23. The method as defined in claim 21, wherein the vitamin is selected from the group consisting of B-complex Vitamins, Vitamin C, Vitamin A, Vitamin D, Vitamin E, Vitamin K and co-enzymes.

24. The method of claim 23, wherein B-complex Vitamins are selected from the group consisting of Vitamin B₁ (thiamine), Vitamin B₂ (riboflavin), Vitamin B₃ (niacin), Vitamin B₅ (pantothenic acid), Vitamin B₆ (pyridoxine), Vitamin B₉ (folic acid), and Vitamin B₁₂ (cyanocobalamin).

25. A method for making a metabolic energy capacity enhancing composition, the method comprising:
selecting an anti-oxidant phytonectar composition;
selecting an energy catalyst blend;
incorporating an anti-oxidant phytonectar and an energy-catalyst blend into a formulation suitable for delivering an effective amount of the anti-oxidants and energy catalysts to a mammal;
administering the formulation to a mammal; and
increasing energy delivery from the diet of the mammal while ameliorating oxidative stress consequent thereto.

26. The method as defined in claim 25, wherein administering the composition further comprises a form selected from the group consisting of liquid, tablet, capsule, powder, granule, spray, ointment and cream.

27. The method as defined in claim 25, wherein the phytonectar further comprises an extract from a plant selected from the group consisting of grape, aloe vera, apple, morinda citrifolia, scullcap, blueberry, prune, cranberry, elderberry, bilberry, gentain, orange, mango, kiwi, pomegranate, green tea, black tea, wheat, blackberry, raspberry, strawberry, onions, pear, cherries, plums, potato, tomato, grapefruit, pineapple, persimmon, eggplant, legume, coffee, guarana, cocoa bean, camellia leaf, kola nut, yerba mate, ginger root, evodia fruit, senna, pau d' arco bark, cascara sagrada, red clover, sheep sorrel, bayberry, boswellia gum, turmeric seed, mangosteen, perilla seed, ginseng root, root beer, stinging nettle leaf, podophyllum, bloodroot, myrrh gum, willow bark, pine bark, echinacea, goldenseal root and devil's claw root.

28. The method as defined in claim 25, wherein the anti-oxidant agent is selected from the group consisting of polyphenol, chelating agent, carotenoid, enzyme, donor anti-oxidant and sacrificial anti-oxidant.

29. The method as defined in claim 28, wherein the anti-oxidant agent further comprises a polyphenol member selected from the group consisting of phenolic acid, flavonoid, stilbene, lignan, xanthine, xingerone, xanthone, anthraquinone, caffeic acid, ferulic acid, tannin, gallic acid, flavone, flavonol, isoflavone, anthocyanin, anthocyanidin, flavanol, proanthocyanidin, flavanone, tannin, boswellic acid, ganistein, daidzein, catechin, baicalin, myricetin, quercetin, resveratrol, bromelain, luteolin, ginsenoside, salicin, pelargonidin, peonidin, cyanidin, delphinidin, malvidin, catechin, epicatechin, epigallocatechin, epicatechingallate and epigallocatechingallate.

30. The method as defined in claim 25, wherein the energy catalyst is selected from the group consisting of mineral, vitamin, co-vitamin, carbohydrate and lipid.

31. The method as defined in claim 30, wherein the mineral is selected from the group consisting of calcium, magnesium, manganese, zinc, chromium, selenium, iron, copper, molybdenum, vanadium, potassium, iodine and cobalt.

32. The method as defined in claim 30, wherein the vitamin is selected from the group consisting of B-complex Vitamins, Vitamin C, Vitamin A, Vitamin D, Vitamin E, Vitamin K and co-enzymes.

33. The method of claim 32, wherein B-complex Vitamins are selected from the group consisting of Vitamin B₁ (thiamine), Vitamin B₂ (riboflavin), Vitamin B₃ (niacin), Vitamin B₅ (pantothenic acid), Vitamin B₆ (pyridoxine), Vitamin B₉ (folic acid), and Vitamin B₁₂ (cyanocobalamin).
